(19) United States
(12) Patent Application Publication
(10) Pub. No.: US 2022/0347071 A1
(43) Pub. Date: Nov. 3, 2022

(54) USE OF CANNABINOIDS IN THE TREATMENT OF ORAL INFLAMMATION AND PERIODONTAL DISEASE

(71) Applicant: Demetrix, Inc., Berkeley, CA (US)

(72) Inventors: Cynthia W. Bryant, Raleigh, NC (US); Alison Watta, Durham, NC (US)

(21) Appl. No.: 17/661,276

(22) Filed: Apr. 28, 2022

Related U.S. Application Data
(60) Provisional application No. 63/181,044, filed on Apr. 28, 2021.

(51) Int. Cl. A61K 8/368; A61K 31/05; A61K 31/192; A61K 45/06; A61K 8/34; A61Q 11/00

(52) U.S. Cl. CPC A61K 8/368; A61K 31/05; A61K 31/192; A61K 45/06; A61K 8/347; A61Q 11/00; A61K 2800/92

(57) ABSTRACT

The present disclosure relates to compositions and methods for reducing, preventing, and/or treating oral conditions comprising the administration of one or more cannabidiols. Specifically, the present disclosure relates to compositions comprising CBG, CBGVA or combinations thereof for the reduction of oral inflammation.

### CROSS-REFERENCE TO RELATED APPLICATIONS

The present disclosure claims the priority benefit of U.S. Provisional Patent App. Ser. No. 63/181,044, filed Apr. 28, 2021, which is hereby incorporated herein by reference.

### TECHNICAL FIELD

The present disclosure relates generally to the use of cannabinoids to treat various oral conditions. The oral conditions including oral inflammation and periodontal disorders.

### BACKGROUND

Oral conditions and diseases are caused or worsened by inflammation. For example, periodontal disease is characterized by chronic inflammation occurring in the surrounding tissues that support the teeth by formation of infected “pockets” or spaces between the teeth and gums. These infected pockets contain debris, predominantly composed of microorganisms and their products (enzymes, endotoxins and other metabolic products), dental plaque, gingival fluid, food remnants, salivary mucin, desquamated epithelial cells, and leukocytes. Periodontal pockets are chronic inflammatory lesions. The destructive changes consist of the fluid and cellular inflammatory exudates and the associated degenerative changes stimulated by local bacterial infiltrate. The constructive changes consist of the formation of connective tissue cells, collagen fibers, and blood vessels in an effort to repair tissue damage caused by the inflammatory process. Healing does not go to completion because of the persistence of local irritants i.e., bacteria and the enzymes that they produce. These irritants stimulate fluid and cellular exudates, which in turn causes degeneration of the new tissue elements formed in the repair process. Interleukin 8 (IL-8) is a pro-inflammatory cytokine and a potent chemoattractant and activator of neutrophils. IL-8 is produced by immune cells (including lymphocytes, neutrophils, monocytes and macrophages), fibroblasts and epithelial cells. Reports indicate an important role for IL-8 in the pathogenesis of many inflammatory disorders, including gingivitis and periodontal disease. An oral care composition that prevents or reduces oral bacteria and/or the release of IL-8 would be effective against periodontal and oral inflammatory diseases or disorders. Additionally, prostaglandin E2 (PGE2 is known to play a role in inflammation. Several studies, including Hussien et al, demonstrated PGE2 levels in gingival crevicular fluid are increased in patients with periodontitis. Reducing levels of PGE2 can be an effective treatment for periodontitis as shown by Sanchez and al.

Accordingly, there is an unmet need for safe and effective oral care compositions that reduce, prevent or treat inflammation-associated infections and conditions, including but not limited to gingivitis and periodontal disease.

### SUMMARY

The present disclosure is directed to a composition for use as an oral care product to reduce, treat, prevent, or ameliorate oral inflammation, comprising cannabigerol (CBG), cannabigerovarinic acid (CBGVA), or a combination thereof.

In some embodiments, the composition comprises an effective amount of CBG, CBGVA, or a combination thereof in an amount effective in reducing, treating, preventing, or ameliorating oral inflammation.

In some embodiments, the composition is an oral care formulation. In some embodiments, the composition is a toothpaste. In some embodiments, the composition is a mouthwash. In some embodiments, the composition is a chewable. In some embodiments, the composition is a wipe. In some embodiments, the composition is a rinse. In some embodiments, the composition is a powder. In some embodiments, the composition is a floss.

In some embodiments, the composition comprises about 0.01 μg/mL to about 0.1 μg/mL, about 0.1 μg/mL to about 1 μg/mL, about 1 μg/mL to about 10 μg/mL, about 10 μg/mL to about 20 μg/mL, about 20 μg/mL to about 30 μg/mL, about 40 μg/mL to about 50 μg/mL, about 50 μg/mL to about 60 μg/mL, about 60 μg/mL to about 70 μg/mL, about 70 μg/mL to about 80 μg/mL, about 80 μg/mL to about 90 μg/mL, about 90 μg/mL to about 100 μg/mL, about 100 μg/mL to about 200 μg/mL, about 200 μg/mL to about 300 μg/mL, about 400 μg/mL to about 500 μg/mL, about 500 μg/mL to about 600 μg/mL, about 600 μg/mL to about 700 μg/mL, about 700 μg/mL to about 800 μg/mL, about 800 μg/mL to about 900 μg/mL, and/or about 900 μg/mL to about 1 mg/mL of CBG.

In some embodiments, the composition comprises about 0.01 μg/mL to about 0.1 μg/mL, about 0.1 μg/mL to about 1 μg/mL, about 1 μg/mL to about 10 μg/mL, about 10 μg/mL to about 20 μg/mL, about 20 μg/mL to about 30 μg/mL, about 40 μg/mL to about 50 μg/mL, about 50 μg/mL to about 60 μg/mL, about 60 μg/mL to about 70 μg/mL, about 70 μg/mL to about 80 μg/mL, about 80 μg/mL to about 90 μg/mL, about 90 μg/mL to about 100 μg/mL, about 100 μg/mL to about 200 μg/mL, about 200 μg/mL to about 300 μg/mL, about 400 μg/mL to about 500 μg/mL, about 500 μg/mL to about 600 μg/mL, about 600 μg/mL to about 700 μg/mL, about 700 μg/mL to about 800 μg/mL, about 800 μg/mL to about 900 μg/mL, and/or about 900 μg/mL to about 1 mg/mL of CBGVA.

In some embodiments, the composition comprises about 0.01 μg/mL to about 0.1 μg/mL, about 0.1 μg/mL to about 1 μg/mL, about 1 μg/mL to about 10 μg/mL, about 10 μg/mL to about 20 μg/mL, about 20 μg/mL to about 30 μg/mL, about 40 μg/mL to about 50 μg/mL, about 50 μg/mL to about 60 μg/mL, about 60 μg/mL to about 70 μg/mL, about 70 μg/mL to about 80 μg/mL, about 80 μg/mL to about 90 μg/mL, about 90 μg/mL to about 100 μg/mL, about 100 μg/mL to about 200 μg/mL, about 200 μg/mL to about 300 μg/mL, about 400 μg/mL to about 500 μg/mL, about 500 μg/mL to about 600 μg/mL, about 600 μg/mL to about 700 μg/mL, about 700 μg/mL to about 800 μg/mL, about 800 μg/mL to about 900 μg/mL, and/or about 900 μg/mL to about 1 mg/mL of CBG and CBGVA, collectively.

In some embodiments, the composition comprises one or more additional oral care active ingredients.

In some embodiments, the composition comprises one or more of cetylpyridinium chloride, stannous fluoride, essential oil, and fluoride ions.

In some embodiments, the composition prevents or reduces the release of interleukin 8 (IL-8).

In some embodiments, the composition prevents or reduces the release of PGE2.

In some embodiments, the composition prevents or reduces the release of both IL-8 and PGE2.

In some embodiments, the composition prevents or reduces one or more condition associated with oral inflammation, the release of IL-8, or the release of PGE2.

In some embodiments, the one or more condition associated with oral inflammation, the release of IL-8, or the release of PGE2 is selected from gingivitis, periodontal disease, periimplantitis, oral mucositis, chemotherapy-induced oral mucositis, and dental pain.

In one aspect, the present disclosure is directed to a method of reducing, treating, preventing, or ameliorating oral inflammation in a subject in need thereof comprising administering an effective amount of the composition of the present disclosure.

In some embodiments, the oral inflammation is associated with or results in gingivitis, periodontal disease, periimplantitis, oral mucositis, chemotherapy-induced oral mucositis, and/or dental pain.

In one aspect, the present disclosure is directed to a method of decreasing the release of IL-8, PGE2, or both in the mouth of a subject in need thereof comprising administering an effective amount of the composition of the present disclosure.

In some embodiments, the composition is substantially free of CBGA. In some embodiments, the composition is substantially free of CBD. In some embodiments, the composition is substantially free of THC.

In one aspect, the present disclosure is directed to a method of making an oral care product comprising adding CBG, CBGVA, or a combination thereof to an oral care formulation.

In one aspect, the present disclosure is directed to an oral care product made by adding CBG, CBGVA, or a combination thereof to an oral care formulation.

In one aspect, the present disclosure is directed to a pharmaceutical composition comprising CBG, CBGVA, or a combination thereof, wherein the pharmaceutical composition comprises an excipient.

### DETAILED DESCRIPTION

### Definitions

As used throughout, ranges are used as shorthand for describing each and every value that it is within the range. Any value within the range can be selected as the terminus of the range.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the term “about”, when applied to the value for a parameter of a composition or method of this invention, indicates that the calculation or the measurement of the value allows some slight imprecision without having a substantial effect on the chemical or physical attributes of the composition or the method. If, for some reason, the imprecision provided by “about” is not otherwise understood in the art with this ordinary meaning, then “about” as used herein indicates a possible variation of up to 5% in the value.

As referred to herein, compositional percentages are by weight of the total composition, unless otherwise specified.

As used herein, “ppm” (parts per million) refers to ppm by weight, unless otherwise indicated.

As referred to herein, all ratios refer to weight ratios, unless otherwise indicated.

As used herein, the term “treating” may refer to, for example, an improvement of one of more symptoms of a condition and/or a delay in disease progression. As used herein, the term “preventing” may refer to, for example, a delay in disease onset compared to, e.g., a population average.

As used herein, the term “subject” may refer to, for example, a patient diagnosed with or suspected of having an oral condition that may benefit from the administration of a composition described herein. The terms “subject” and “patient” are used interchangeably herein. In some embodiments, the subject is human. In some embodiments, the subject is a human adult, e.g., is over 18 years of age, about 18-30 years of age, about 30-40 years of age, about 40-45 years of age, about 45-50 years of age, about 50-55 years of age, about 55-60 years of age, about 60-65 years of age, about 65-70 years of age, about 70-75 years of age, about 75-80 years of age, about 80-85 years of age, about 85-90 years of age, or over 90 years of age.

The term “one or more cannabinoid” generally refers to CBG either alone or in combination with one or more as discussed herein. The term “one or more cannabinoid” generally refers to CBGVA either alone or in combination with one or more as discussed herein.

### Compositions

The present disclosure describes use of cannabinoids to prevent, reduce or treat various oral conditions.

It should be understood that the examples and embodiments described in this disclosure, are intended for illustration only and are not intended to limit the scope of the invention. As used herein, ranges are used as shorthand for describing each and every value that it is within the range, as well as the values indicating the minimum and maximum of a range. Any value within the range can also be selected as the terminus of the range.

In one aspect, the disclosure describes compositions useful in the prevention or reduction of oral inflammation. By preventing the release of IL-8, PGE2 or both, there would be a reduction or prevention of oral inflammation. Accordingly, the present disclosure provides, in one aspect, compositions for use as oral care products, where the composition includes cannabigerol (CBG), cannabigerovarinic acid (CBGVA), or combinations thereof in an amount effective to prevent or reduce the release of IL-8, PGE2 or both. In one embodiment, the composition prevents or reduces oral inflammation. The composition may prevent, reduce, or treat periodontal disease.

In some variations, the composition is an oral care formulation, such as a toothpaste, mouthwash, chewable, wipe, rinse, powder, or floss. In another aspect, the disclosure provides methods of making an oral care product by adding the appropriate cannabinoid, and an oral care product made by such methods.

It should be understood that the detailed description and specific examples, whilst indicating embodiments of the invention, are intended for the purpose of illustration only and are not intended to limit the scope of the invention.

The cannabinoid combinations as discussed herein and compositions comprising the same can be used to treat one or more oral conditions within the mouth. “Mouth” as used herein refers to the cavity bounded externally by the lips and internally by the pharynx that encloses the tongue, gums and teeth. Thus, the tissues of the mouth include the lips, tongue, gums, buccal tissue, palate and teeth. A single tissue, a plurality of tissues, a portion of one or more tissues, all or substantially all of the tissues of the mouth, or combinations of the foregoing, may be treated according to the invention.

As used herein, “treat” and variations thereof as used herein refers to cure, ameliorate, alleviate, inhibit, prevent, reduce the likelihood of, or reduce the severity of, a disease or condition, or of at least some of the symptoms or effects thereof. To treat a tissue of the mouth, the tissue is contacted with the composition effective at treating the condition. In some embodiments, oral inflammation may be contacted with an oral care composition comprising CBG, CBGVA, or a combination thereof.

In some embodiments, the compositions herein can be used in combination with one or more of cannabidiol (CBD), cannabidiolic acid (CBDA), cannabidivarin (CBDV), cannabigervarin (CBGV) and tetrahydrocannabivarin (THCV).

### Compositions and Methods for Treating Oral Inflammation and Associated Conditions

The present disclosure provides compositions and methods for treating, preventing, reducing, or ameliorating inflammation in a tissue of a subject's mouth. In some embodiments, the composition of the present disclosure inhibits IL-8 production. In some embodiments, the composition of the present disclosure treats or prevents chronic periodontitis by the inhibition of IL-8 production. Diseases and conditions treatable according to the invention include inflammation and inflammatory disease and conditions, such as gingivitis and periodontitis, and any disease or condition involving, caused by, or exacerbated by, inflammation, IL-8 release

### Periodontal Disease

Periodontal disease is characterized by inflammation occurring in the tissues that support the teeth. The inflamed tissue contains debris, predominantly composed of microorganisms and their products (enzymes, endotoxins and other metabolic products), dental plaque, gingival fluid, food remnants, salivary mucin, desquamated epithelial cells, and leukocytes. Healing does not go to completion because of the persistence of local irritants i.e., bacteria and the enzymes that they produce. These irritants stimulate fluid and cellular exudates, which in turn causes degeneration of the new tissue elements formed in the repair process. Interleukin 8 (IL-8) is a pro-inflammatory cytokine and a potent chemoattractant and activator of neutrophils which plays an important role in the pathogenesis of many inflammatory disorders, including gingivitis and periodontal disease. Sfakianakis et al., J. Periodontal Res., 37(2):154-160 (April 2002), Fitzgerald et al., Oral Microbiol. Immunol., 10(5):297-303 (October 1995), and Takigawa et al., J. Periodontol., 65(11): 1002-1007 (November 1994).

In one aspect, the present disclosure provides compositions and methods of inhibiting the release of pro-inflammatory cytokines (e.g. IL-8) from cells located in a tissue of a subject's mouth. In some embodiments, the oral care composition treats one or more inflammation-based conditions. In some embodiments, the inflammation-based condition to be treated, prevented, reduced, or ameliorated in accordance with the compositions or methods described herein is periodontal disease or gingivitis.

Without being bound by any theory, a decrease in expression of PGE2 and/or IL-8 results in decreased inflammation. As shown in Example 1, the compositions described herein are effective in decreasing the release of IL-8 and PGE-2. In some embodiments, a method of treating, preventing, reducing, or ameliorating oral inflammation, or a periodontal condition or disease described herein results in decreased PGE2 expression and/or decreased PGE2 signaling. In some embodiments, a method of treating, preventing, reducing, or ameliorating oral inflammation or a periodontal condition or disease described herein results in decreased expression of inflammatory cytokines (e.g., IL-8). The outcome of a method of treating, preventing, reducing, or ameliorating oral inflammation or a periodontal condition or disease described herein may be determined by studying a biochemical marker of inflammation, for example, PGE2 expression or expression of inflammatory cytokines (e.g., IL-8). Expression of PGE2 and inflammatory cytokines may be determined using any suitable method known in the art or described herein. For example, protein expression may be measured by Enzyme-linked Immunosorbent Assay (ELISA), Western Blotting, or immunofluorescence, while gene expression maybe measured by quantitative real-time PCR or RNA sequencing.

In some embodiments, a composition provided herein for the treatment of oral inflammation and associated conditions is substantially free of cannabinoids other than CBG and/or CBGVA. For example, the composition may comprise less than 5%, less than 4%, less than 3%, less than 2%, less than 1%, less than 0.5%, or less than 0.1% cannabinoids other than CBG and/or CBGVA. “Cannabinoids” include, without limitation, CBG, CBGVA, cannabidiol (CBD), cannabigerolic acid (CBGA), and cannabidiolic acid (CBDA).

Thus, in some embodiments, the composition described herein is substantially free of CBD. In some embodiments, the composition described herein is substantially free of CBGA. In some embodiments, the composition described herein is substantially free of CBDA. In some embodiments, the composition described herein is substantially free of CBD and CBGA. In some embodiments, the composition described herein is substantially free of CBD and CBDA. In some embodiments, the composition described herein is substantially free of CBGA and CBDA. In some embodiments, the composition described herein is substantially free of CBGA, CBDA, and CBD. In some embodiments, the composition described herein is substantially free of THC.

Prior to the present discovery, it was not appreciated which specific cannabinoid compounds could act to treat oral inflammation and associated conditions. By selecting the cannabinoid compounds with the greatest effectiveness against oral inflammation, the present disclosure provides improved methods of improving oral health.

The present discovery was facilitated by the Applicant's unique methods of producing hereto rare cannabinoid compounds in appreciable quantities including through chemical synthesis reactions and growth in yeast cultures. Prior to the Applicant's research, the lack of viable production of individual cannabinoid compounds obviated the ability to treat oral conditions with specific cannabinoid compounds. Additional details about the production of producing rare cannabinoid compounds are described in PCT Patent Application Nos. WO 2020/069142 A1, WO 2020/069214 A2, WO 2021/05597 A1; and WO 2020/236789 A1, each of which is incorporated herein by reference.

Prior to the Applicant's process of isolating specific and unique cannabinoid compounds from non-horticultural sources, cannabinoid compounds were extracted and isolated only from naturally grown marijuana plants which drastically limited the volume of the rarer cannabinoid compounds available for research or use. Thus, these non-horticulturally derived cannabinoid compounds offer benefits in regard to the treatment of oral inflammation not previously contemplated. As used herein, non-horticulturally derived cannabinoid compounds refers to cannabinoid compounds not grown in plants (e.g., not through horticulture or agriculture).

Additionally, isolated cannabinoid compounds extracted from marijuana plants can also suffer from purity issues as certain unavoidable containments (such as other natural marijuana plant compounds, irremovable amounts of other cannabinoid compounds, etc.) can remain present in isolated cannabinoid compounds extracted from marijuana plants. Such unavoidable containments can impact the quality of the data or even alter the apparent functioning of the cannabinoid compounds. Compositions and methods of treating oral inflammation that use horticulturally derived cannabinoid compounds may not exhibit the same effects as compositions and methods using purer cannabinoid compounds such as the cannabinoid compounds contemplated herein. As can be appreciated however, horticulturally derived cannabinoid compounds can be used in certain embodiments of the disclosure if the horticulturally derived cannabinoid compounds are sufficiently pure and/or if any containments are sufficiently well understood.

In some embodiments, the composition is a natural product, e.g., an extract of a cannabis plant. In some embodiments, the composition is a concentrated extract of a plant belonging to the cannabis genus. In some embodiments, the composition is a synthetic product. In some embodiments, the cannabinoids discussed herein are produced via fermentation.

In some embodiments, the composition of the present disclosure comprises CBG at a concentration of at most about 5 μg/mL, at most about 10 μg/mL, at most about 25 μg/mL, at most about 50 μg/mL, at most about 100 μg/mL, at most about 200 μg/mL, at most about 400 μg/mL, at most about 800 μg/mL, or at most about 1600 μg/mL.

In some embodiments, the composition of the present disclosure comprises CBG at a concentration of at least about 5 μg/mL, at least about 10 μg/mL, at least about 25 μg/mL, at least about 50 μg/mL, at least about 100 μg/mL, at least about 200 μg/mL, at least about 400 μg/mL, at least about 800 μg/mL, or at least about 1600 μg/mL.

In some embodiments, the composition comprises about 0.01 μg/mL to about 0.1 μg/mL, about 0.1 μg/mL to about 1 μg/mL, about 1 μg/mL to about 10 μg/mL, about 10 μg/mL to about 20 μg/mL, about 20 μg/mL to about 30 μg/mL, about 40 μg/mL to about 50 μg/mL, about 50 μg/mL to about 60 μg/mL, about 60 μg/mL to about 70 μg/mL, about 70 μg/mL to about 80 μg/mL, about 80 μg/mL to about 90 μg/mL, about 90 μg/mL to about 100 μg/mL, about 100 μg/mL to about 200 μg/mL, about 200 μg/mL to about 300 μg/mL, about 400 μg/mL to about 500 μg/mL, about 500 μg/mL to about 600 μg/mL, about 600 μg/mL to about 700 μg/mL, about 700 μg/mL to about 800 μg/mL, about 800 μg/mL to about 900 μg/mL, and/or about 900 μg/mL to about 1 mg/mL of CBG.

In some embodiments, the composition provided herein comprises about 5-10% (w/w), about 10-20% (w/w), about 20-30% w/w, about 30-40% w/w, about 50-60% w/w, about 60-70% w/w, about 70-80% w/w, about 80-90% w/w, and/or more than 90% w/w of CBG.

In some embodiments, the composition of the present disclosure comprises CBGVA at a concentration of at most about 5 μg/mL, at most about 10 μg/mL, at most about 25 μg/mL, at most about 50 μg/mL, at most about 100 μg/mL, at most about 200 μg/mL, at most about 400 μg/mL, at most about 800 μg/mL, or at most about 1600 μg/mL.

In some embodiments, the composition of the present disclosure comprises CBGVA at a concentration of at least about 5 μg/mL, at least about 10 μg/mL, at least about 25 μg/mL, at least about 50 μg/mL, at least about 100 μg/mL, at least about 200 μg/mL, at least about 400 μg/mL, at least about 800 μg/mL, or at least about 1600 μg/mL.

In some embodiments, the composition comprises about 0.01 μg/mL to about 0.1 μg/mL, about 0.1 μg/mL to about 1 μg/mL, about 1 μg/mL to about 10 μg/mL, about 10 μg/mL to about 20 μg/mL, about 20 μg/mL to about 30 μg/mL, about 40 μg/mL to about 50 μg/mL, about 50 μg/mL to about 60 μg/mL, about 60 μg/mL to about 70 μg/mL, about 70 μg/mL to about 80 μg/mL, about 80 μg/mL to about 90 μg/mL, about 90 μg/mL to about 100 μg/mL, about 100 μg/mL to about 200 μg/mL, about 200 μg/mL to about 300 μg/mL, about 400 μg/mL to about 500 μg/mL, about 500 μg/mL to about 600 μg/mL, about 600 μg/mL to about 700 μg/mL, about 700 μg/mL to about 800 μg/mL, about 800 μg/mL to about 900 μg/mL, and/or about 900 μg/mL to about 1 mg/mL of CBGVA.

In some embodiments, the composition provided herein comprises about 5-10% (w/w), about 10-20% (w/w), about 20-30% w/w, about 30-40% w/w, about 50-60% w/w, about 60-70% w/w, about 70-80% w/w, about 80-90% w/w, and/or more than 90% w/w of CBGVA.

In some embodiments, the composition of the present disclosure comprises CBG and CBGVA in a combined concentration of at most about 5 μg/mL, at most about 10 μg/mL, at most about 25 μg/mL, at most about 50 μg/mL, at most about 100 μg/mL, at most about 200 μg/mL, at most about 400 μg/mL, at most about 800 μg/mL, or at most about 1600 μg/mL.

In some embodiments, the composition of the present disclosure comprises CBG and CBGVA in a combined concentration of at least about 5 μg/mL, at least about 10 μg/mL, at least about 25 μg/mL, at least about 50 μg/mL, at least about 100 μg/mL, at least about 200 μg/mL, at least about 400 μg/mL, at least about 800 μg/mL, or at least about 1600 μg/mL.

In some embodiments, the composition comprises about 0.01 μg/mL to about 0.1 μg/mL, about 0.1 μg/mL to about 1 μg/mL, about 1 μg/mL to about 10 μg/mL, about 10 μg/mL to about 20 μg/mL, about 20 μg/mL to about 30 μg/mL, about 40 μg/mL to about 50 μg/mL, about 50 μg/mL to about 60 μg/mL, about 60 μg/mL to about 70 μg/mL, about 70 μg/mL to about 80 μg/mL, about 80 μg/mL to about 90 μg/mL, about 90 μg/mL to about 100 μg/mL, about 100 μg/mL to about 200 μg/mL, about 200 μg/mL to about 300 μg/mL, about 400 μg/mL to about 500 μg/mL, about 500 μg/mL to about 600 μg/mL, about 600 μg/mL to about 700 μg/mL, about 700 μg/mL to about 800 μg/mL, about 800 μg/mL to about 900 μg/mL, and/or about 900 μg/mL to about 1 mg/mL of both CBG and CBGVA, collectively.

In some embodiments, the composition provided herein comprises about 5-10% (w/w), about 10-20% (w/w), about 20-30% w/w, about 30-40% w/w, about 50-60% w/w, about 60-70% w/w, about 70-80% w/w, about 80-90% w/w, and/or more than 90% w/w of both CBG and CBGVA, collectively.

In some embodiments, the composition of the present disclosure comprises CBG, CBGVA, or a combination thereof at a concentration of between at least about 0.1 μM and 1 mM. In some embodiments, the composition of the present disclosure comprises CBG, CBGVA, or a combination thereof at a concentration of between at least about 0.5 μM and 1 mM, between at least about 1 μM and 1 mM, between at least about 2 μM and 1 mM, between at least about 5 μM and 1 mM, between at least about 10 μM and 1 mM, between at least about 15 μM and 1 mM, between at least about 20 μM and 1 mM, between at least about 25 μM and 1 mM, between at least about 50 μM and 1 mM, between at least about 100 μM and 1 mM, between at least about 150 μM and 1 mM, between at least about 200 μM and 1 mM, between at least about 250 μM and 1 mM, between at least about 300 μM and 1 mM, between at least about 350 μM and 1 mM, between at least about 400 μM and 1 mM, between at least about 450 μM and 1 mM, or between at least about 500 μM and 1 mM.

In some embodiments, the composition of the present disclosure comprises CBG, CBGVA, or a combination thereof at a concentration of between at least about 0.1 μM and 500 μM. In some embodiments, the composition of the present disclosure comprises CBG, CBGVA, or a combination thereof at a concentration of between at least about 0.5 μM and 500 μM, between at least about 1 μM and 500 μM, between at least about 2 μM and 500 μM, between at least about 5 μM and 500 μM, between at least about 10 μM and 500 μM, between at least about 15 μM and 500 μM, between at least about 20 μM and 500 μM, between at least about 25 μM and 500 μM, between at least about 50 μM and 500 μM, between at least about 100 μM and 500 μM, between at least about 150 μM and 500 μM, between at least about 200 μM and 500 μM, between at least about 250 μM and 500 μM, between at least about 300 μM and 500 μM, between at least about 350 μM and 500 μM, between at least about 400 μM and 500 μM, or between at least about 450 μM and 500 μM.

In some embodiments, the composition of the present disclosure comprises CBG, CBGVA, or a combination thereof at a concentration of between at least about 0.1 μM and 250 μM. In some embodiments, the composition of the present disclosure comprises CBG, CBGVA, or a combination thereof at a concentration of between at least about 0.5 μM and 250 μM, between at least about 1 μM and 250 μM, between at least about 2 μM and 250 μM, between at least about 5 μM and 250 μM, between at least about 10 μM and 250 μM, between at least about 15 μM and 250 μM, between at least about 20 μM and 250 μM, between at least about 25 μM and 250 μM, between at least about 50 μM and 250 μM, between at least about 100 μM and 250 μM, between at least about 150 μM and 250 μM, or between at least about 200 μM and 250 μM.

In some embodiments, the composition of the present disclosure comprises CBG, CBGVA, or a combination thereof at a concentration of between at least about 0.1 μM and 100 μM. In some embodiments, the composition of the present disclosure comprises CBG, CBGVA, or a combination thereof at a concentration of between at least about 0.5 μM and 100 μM, between at least about 1 μM and 100 μM, between at least about 2 μM and 100 μM, between at least about 5 μM and 100 μM, between at least about 10 μM and 100 μM, between at least about 15 μM and 100 μM, between at least about 20 μM and 100 μM, between at least about 25 μM and 100 μM, or between at least about 50 μM and 100 μM.

In some embodiments, the composition of the present disclosure comprises CBG, CBGVA, or a combination thereof at a concentration of between at least about 0.1 μM and 75 μM. In some embodiments, the composition of the present disclosure comprises CBG, CBGVA, or a combination thereof at a concentration of between at least about 0.5 μM and 75 μM, between at least about 1 μM and 75 μM, between at least about 2 μM and 75 μM, between at least about 5 μM and 75 μM, between at least about 10 μM and 75 μM, between at least about 15 μM and 75 μM, between at least about 20 μM and 100 μM, between at least about 25 μM and 75 μM, or between at least about 50 μM and 75 μM.

In some embodiments, the composition of the present disclosure comprises CBG, CBGVA, or a combination thereof at a concentration of between at least about 0.1 μM and 50 μM. In some embodiments, the composition of the present disclosure comprises CBG, CBGVA, or a combination thereof at a concentration of between at least about 0.5 μM and 50 μM, between at least about 1 μM and 50 μM, between at least about 2 μM and 50 μM, between at least about 5 μM and 50 μM, between at least about 10 μM and 50 μM, between at least about 15 μM and 50 μM, between at least about 20 μM and 50 μM, or between at least about 25 μM and 50 μM.

In some embodiments, the composition of the present disclosure comprises CBG, CBGVA, or a combination thereof at a concentration of between at least about 0.1 μM and 25 μM. In some embodiments, the composition of the present disclosure comprises CBG, CBGVA, or a combination thereof at a concentration of between at least about 0.5 μM and 25 μM, between at least about 1 μM and 25 μM, between at least about 2 μM and 25 μM, between at least about 5 μM and 25 μM, between at least about 10 μM and 25 μM, between at least about 15 μM and 25 μM, or between at least about 20 μM and 25 μM.

In some embodiments, the composition of the present disclosure comprises CBG, CBGVA, or a combination thereof at a concentration of between at least about 0.1 μM and 20 μM. In some embodiments, the composition of the present disclosure comprises CBG, CBGVA, or a combination thereof at a concentration of between at least about 0.5 μM and 20 μM, between at least about 1 μM and 20 μM, between at least about 2 μM and 20 μM, between at least about 5 μM and 20 μM, between at least about 10 μM and 20 μM, or between at least about 15 μM and 20 μM.

In some embodiments, the composition of the present disclosure comprises CBG, CBGVA, or a combination thereof at a concentration of between at least about 0.1 μM and 15 μM. In some embodiments, the composition of the present disclosure comprises CBG, CBGVA, or a combination thereof at a concentration of between at least about 0.5 μM and 15 μM, between at least about 1 μM and 15 μM, between at least about 2 μM and 15 μM, between at least about 5 μM and 15 μM, between at least about 10 μM and 15 μM.

In some embodiments, the composition of the present disclosure comprises CBG, CBGVA, or a combination thereof at a concentration of between at least about 0.1 μM and 10 μM. In some embodiments, the composition of the present disclosure comprises CBG, CBGVA, or a combination thereof at a concentration of between at least about 0.5 μM and 10 μM, between at least about 1 μM and 10 μM, between at least about 2 μM and 10 μM, between at least about 5 μM and 10 μM.

In some embodiments, the composition of the present disclosure comprises CBG, CBGVA, or a combination thereof at a concentration of between at least about 0.1 μM and 5 μM. In some embodiments, the composition of the present disclosure comprises CBG, CBGVA, or a combination thereof at a concentration of between at least about 0.5 μM and 5 μM, between at least about 1 μM and 5 μM, between at least about 2 μM and 5 μM.

In some embodiments, the composition of the present disclosure comprises CBG, CBGVA, or a combination thereof at a concentration of between at least about 0.1 μM and 2 μM. In some embodiments, the composition of the present disclosure comprises CBG, CBGVA, or a combination thereof at a concentration of between at least about 0.5 μM and 2 μM, between at least about 1 μM and 2 μM.

In some embodiments, the composition of the present disclosure comprises CBG, CBGVA, or a combination thereof at a concentration of between at least about 0.1 μM and 1 μM. In some embodiments, the composition of the present disclosure comprises CBG, CBGVA, or a combination thereof at a concentration of between at least about 0.5 μM and 1 μM.

In some embodiments, the composition of the present disclosure comprises CBG, CBGVA, or a combination thereof at a concentration of between at least about 0.1 μM and 0.5 μM.

In some embodiments, the composition of the present disclosure comprises a combination of CBG and CBVGA.

In some embodiments, the combination is a 9:1 CBG:CBGVA ratio.

In some embodiments, the combination is a 8:1 CBG:CBGVA ratio.

In some embodiments, the combination is a 7:1 CBG:CBGVA ratio.

In some embodiments, the combination is a 6:1 CBG:CBGVA ratio.

In some embodiments, the combination is a 5:1 CBG:CBGVA ratio.

In some embodiments, the combination is a 4:1 CBG:CBGVA ratio.

In some embodiments, the combination is a 3:1 CBG:CBGVA ratio.

In some embodiments, the combination is a 2:1 CBG:CBGVA ratio.

In some embodiments, the combination is a 1:1 CBG:CBGVA ratio.

In some embodiments, the combination is a 1:2 CBG:CBGVA ratio.

In some embodiments, the combination is a 1:3 CBG:CBGVA ratio.

In some embodiments, the combination is a 1:4 CBG:CBGVA ratio.

In some embodiments, the combination is a 1:5 CBG:CBGVA ratio.

In some embodiments, the combination is a 1:6 CBG:CBGVA ratio.

In some embodiments, the combination is a 1:7 CBG:CBGVA ratio.

In some embodiments, the combination is a 1:8 CBG:CBGVA ratio.

In some embodiments, the combination is a 1:9 CBG:CBGVA ratio.

### Oral Care Compositions

A wide variety of different oral care compositions can be prepared utilizing one or more cannabinoids of the present disclosure using materials and methods known in the art or which will be developed. It is within the skill in the art to choose appropriate ingredients and combinations of ingredients and to determine the specific amount of one or more cannabinoids to include in a particular oral care composition, given the knowledge in the art and the guidance provided herein.

Oral care compositions of the present disclosure include washes, rinses, gargles, solutions, drops, emulsions, suspensions, liquids, pastes, gels, ointments, creams, sprays, powders, tablets, gums, lozenges, mints, films, patches, and tooth whitening compositions. Oral care compositions of the invention include compositions intended for use by consumers and patients and compositions intended for use by dental professionals (e.g., dental hygienists, dentists and oral surgeons).

One or more cannabinoids can be incorporated into an oral care composition or device, such as a toothpaste, a tooth gel, powder, wipe, a mouthwash or rinse, or a dental floss. In some embodiments, one or more cannabinoids may be contained in a separate oral care composition or device which will be used separately from other compositions and devices employed in the prophylactic oral care regimen. For instance, one or more cannabinoids can be incorporated into a mouthwash or rinse, a gum, a lozenge or a chewable tablet.

In some embodiments, dentifrices include toothpastes, tooth gels, tooth powders and liquid dentifrices. Toothpastes and tooth gels generally include a dental abrasive, a surfactant, a thickening agent, a humectant, a flavoring agent, a sweetening agent, a coloring agent and water. Toothpastes and tooth gels may also include opacifying agents, anti-caries agents, anti-calculus agents, tooth whitening agents, and other optional ingredients. Liquid dentifrices may comprise water, ethanol, a humectant, a surfactant, a thickening agent, an abrasive, an anti-caries agent, a flavoring agent and a sweetening agent.

In some embodiments, gels include dentifrice gels, non-abrasive gels and subgingival gels. Non-abrasive gels and subgingival gels generally include a thickening agent, a humectant, a flavoring agent, a sweetening agent, a coloring agent, and water. Such gels may also include one or more anti-caries agents and/or anti-calculus agents.

In some embodiments, creams generally include a thickening agent, a humectant and a surfactant, and may include a flavoring agent, a sweetening agent, a coloring agent.

In some embodiments, ointments suitable for oral use generally include one or more of the following: fats, oils, waxes, paraffins, silicones, pastises, alcohols, water, humectants, surfactants, thickening agents, talc, bentonites, zinc oxide, aluminum compounds, preservatives, antiviral compounds, and other ingredients.

In some embodiments, mouthwashes, rinses, gargles and sprays generally include water, ethanol, and/or a humectant, and may also include a surfactant, a flavoring agent, a sweetening agent, and a coloring agent, and may include a thickening agent and one or more anti-caries agents and/or anti-calculus agents.

Oral care devices of the invention include materials (such as sutures and sponges), flosses, tapes, chips, strips, fibers, a toothpick or rubber tip, syringes, dental implants and dental appliances (such as trays and troughs that fit over and cover the teeth and, optionally, the periodontal tissue) having one or more cannabinoids adhered to, absorbed into, bound to, attached to, entrapped in, enclosed in, coated onto, or otherwise incorporated into, them.

As one example, a compressed chewable tablet comprises a water-disintegrable, compressible carbohydrate (such as mannitol, sorbitol, maltitol, dextrose, sucrose, xylitol, lactose and mixtures thereof), a binder (such as cellulose, cellulosic derivatives, polyvinyl pyrrolidone, starch, modified starch and mixtures thereof), and, optionally, a lubricant (such as magnesium stearate, stearic acid, talc, and waxes), sweetening, coloring and flavoring agents, a surfactant, a preservative, and other ingredients. All of the ingredients, including one or more cannabinoids, are dry blended and compressed into a tablet.

The use of tooth whitening compositions, or of one of the many oral care compositions and devices which comprise a tooth whitening agent, results in the production of ROS and can cause inflammation of the tissues of the mouth. Incorporation of one or more cannabinoids in tooth whitening compositions or other oral care compositions and devices comprising a tooth whitening agent will reduce or prevent the inflammation caused by the production of ROS. An oral care composition or device comprising one or more cannabinoids can be used before or after the tooth whitening composition or oral care composition or device comprising a tooth whitening agent to reduce or prevent the inflammation from the production of ROS. For instance, teeth are commonly whitened by applying a tooth whitening composition to the teeth by means of a dental tray or trough. One or more cannabinoids could be incorporated into the tooth whitening composition that is used in the tray or trough. Alternatively, a separate composition comprising one or more cannabinoids could be applied to the teeth in a cleaned or different tray or trough after the application of the tooth whitening composition is completed. In a further alternative, a wash or rinse comprising one or more cannabinoids could be used to rinse the mouth before and/or after the application of the tooth whitening composition. Another composition for applying a tooth whitening composition to the teeth is a flexible strip. One or more cannabinoids according to the present disclosure could be incorporated into such strips.

Chewing gum compositions generally include a gum base, a flavoring agent and a sweetening agent. Suitable gum bases include jelutong, rubber, latex, chicle, and vinylite resins, desirably with conventional plasticizers or softeners. Plasticizers include triacetin, acetyl tributyl citrate, diethyl sebacetate, triethyl citrate, dibutyl sebacetate, dibutyl succinate, diethyl phthalate and acetylated monoglycerides. Typically, chewing gum compositions contain from about 50% to about 99% gum base, from about 0.4% to about 2% of a flavoring agent and from about 0.01% to about 20% of a sweetening agent. One or more cannabinoids may be incorporated into a gum base by, e.g., stirring them into a warm gum base or coating them onto the outer surface of the gum base.

In some embodiments, additional ingredients may be added to the oral care composition of the present disclosure. Conventional ingredients used in oral care compositions include water, alcohols, humectants, surfactants, thickening agents, abrasives, flavoring agents, sweetening agents, antimicrobial agents, anti-caries agents, anti-plaque agents, anti-calculus agents, pH-adjusting agents, and many others.

Humectants suitable for use in oral care compositions include edible polyhydric alcohols such as glycerol, sorbitol, xylitol, butylene glycol, polyethylene glycol, propylene glycol, mannitol, and lactitol. Humectants help keep oral care compositions, such as pastes, from hardening upon exposure to air, give oral care compositions a moist feel to the mouth, and may impart desirable sweetness.

Surfactants include anionic, nonionic, amphoteric, zwitterionic and cationic synthetic detergents. Anionic surfactants include the water-soluble salts of alkyl sulfates having 8-20 carbon atoms in the alkyl radical (such as sodium alkyl sulfate), the water-soluble salts of sulfonated monoglycerides of fatty acids having from 8-20 carbon atoms (such as sodium lauryl sulfate and sodium coconut monoglyceride sulfonates), sarcosinates (such as sodium and potassium salts of lauroyl sarcosinate, myristoyl sarcosinate, palmitoyl sarcosinate, stearoyl sarcosinate and oleoyl sarcosinate), taurates, higher alkyl sulfoacettes (such as sodium lauryl sulfoacetate), isethionates (such as sodium lauroyl isethionate), sodium laureth carboxylate, sodium dodecyl benezesulfonate, and mixtures of the foregoing. Preferred are the sarcosinates since they inhibit acid formation in the mouth due to carbohydrate breakdown. Nonionic surfactants include poloxamers (sold under the tradename Pluronic), polyoxyethylene sorbitan esters (sold under the tradename Tween), fatty alcohol ethoxylates, polyethylene oxide condensates of alkyl phenols, products derived from the condensation of ethylene oxide with fatty acids, fatty alcohols, fatty amides, polyhydric alcohols, and polypropyleneoxide, ethylene oxide condensates of aliphatic alcohols, long-chain tertiary amine oxides, long-chain tertiary phospine oxides, long-chain dialkyl sulfoxides, and mixtures of such materials. Amphoteric surfactants include betaines (such as cocamidopropylbetaine), derivatives of aliphatic secondary and tertiary amines in which the aliphatic radical can be a straight or branched chain and wherein one of the aliphatic substituents contains about 8-18 carbon atoms and one contains an anionic water-solubilizing group (such as carboxylate, sulfonate, sulfate, phosphate or phosphonate), and mixtures of such materials. Zwitterionic surfactants include derivatives of aliphatic quaternary ammonium, phosphonium and sulfonium compounds in which the aliphatic radical can be a straight or branched chain and wherein one of the aliphatic substituents contains about 8-18 carbon atoms and one contains an anionic water-solubilizing group (such as carboxy, sulfonate, sulfate, phosphate or phosphonate). Cationic surfactants include aliphatic quaternary ammonium compounds having one long alkyl chain containing about 8-18 carbon atoms (such as lauryl trimethylammonium chloride, cetyltrimethylammonium bromide, diisobuytylphenoxyethyldimethylbenzylammonium chloride, coconut alkyltrimetylammonium nitrite, cetylpyridinium fluoride). Certain cationic surfactants can al so act as antimicrobials.

Thickening agents include carboxyvinyl polymers, polyvinylpyrrolidone, polyacrylates, carrageenan, cellulose derivatives (e.g., hydroxypropyl cellulose, hydroxypropyl methyl cellulose, methyl cellulose, and hydroxyethyl cellulose), laponite, water-soluble salts of cellulose ethers (such as sodium carboxymethylcellulose and sodium carboxymethyl hydroxyethyl cellulose), natural gums (such as gum karaya, xanthan gum, gum arabic and gum tragacanth), polymeric polyether compounds (such as polyethylene oxide and polypropylene oxide), homopolymers of acrylic acid crosslinked with an alkyl ether of pentaerythritol, alkyl ether of sucrose, carbomers (sold under the tradename Carbopol®, starch, copolymers of lactide and glycolide monomers (the copolymer having an average molecular weight of about 1,000-120,000), colloidal magnesium aluminum silicate and finely divided silica. Thickening agents will be added in amounts sufficient to give a desired consistency to an oral care composition.

Abrasives include silicas (including gels and precipitates), aluminas, calcium carbonates, calcium phosphates, dicalcium phosphates, tricalcium phosphates, hydroxyapatites, calcium pyrophosphates, trimetaphosphates, insoluble polymetaphopsphates (such as insoluble sodium polymetaphosphate and calcium polymetaphosphate), magnesium carbonates, magnesium oxides, resinous abrasive materials (such as particulate condensation products of urea and formaldehyde), particulate thermosetting polymerized resins (suitable resins include melamines, phenolics, ureas, melamine-ureas, melamine-formaldehydes, urea-formaldehydes, melamine-urea-formaldehydes, cross-linked epoxides and cross-linked polyesters), and combinations of the foregoing. Silica abrasives are preferred because they provide excellent dental cleaning and polishing performance without unduly abrading tooth enamel or dentine.

Flavoring agents include peppermint, oil, spearmint oil, wintergreen oil, clove, menthol, dihydroanethole, estragole, methyl salicylate, eucalyptol, cassia, 1-menthyl acetate, sage, eugenol, parsley oil, menthone, oxanone, alpha-irisone, alpha-ionone, anise, marjoram, lemon, orange, propenyl guaethol, cinnamon, vanillin, ethyl vanillin, thymol, linalool, limonene, isoamylacetate, benzaldehyde, ethylbutyrate, phenyl ethyl alcohol, sweet birch, cinnamic aldehyde, cinnamaldehyde glycerol acetal (known as CGA), and mixtures of the foregoing.

Sweetening agents include sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame, cyclamate salts, and mixtures of the foregoing.

In addition to the flavoring and sweetening agents, the oral care compositions may include coolants, salivating agents, warming agents and numbing agents as optional ingredients. Coolants include carboxamides, menthol, paramenthan carboxamides, isopropylbutanamide, ketals, diols, 3-1-menthoxypropane-1,2-diol, menthone glycerol acetal, menthyl lactate, and mixtures thereof. Salivating agents include Jambu® (manufactured by Takasago). Warming agents include capsicum and nicotinate esters (such as benzyl nicotinate). Numbing agents include benzocaine, lidocaine, clove bud oil and ethanol.

Anti-caries agents include sodium fluoride, stannous fluoride, potassium fluoride, amine fluorides, indium fluoride, sodium monofluorophosphate, calcium lactate, calcium glycerophosphates, strontium salts, and strontium polyacrylates.

Anti-calculus agents include pyrophosphate salts such as dialkali metal pyrophosphate salts and tetraalkali metal pyrophosphate salts (e.g., disodium dihydrogen pyrophosphate, tetrasodium pyrophosphate and tetrapotassium pyrophosphate, in their hydrated and unhydrated forms). Other anti-calculus agents which can be used instead of, or in addition to, the pyrophosphate salts include synthetic anionic polymers (such as polyacrylates and copolymers of maleic anhydride or acid and methyl vinyl ether), polyaminopropane sulfonic acid, zinc citrate trihydrate, polyphosphates (such as tripolyphosphate and hexametaphosphate), polyphosphonates (such as disodium ethane-1-hydroxy-1,1-diphosphonate (EHDP), methanedisphosphonic acid, and 2-phosphonobutane-1,2,4-tricarboxylic acid), and polypeptides (such as polyaspartic acid and polyglutamic acid).

A pH-adjusting agent and/or a buffering agent or agents may need to be included in the oral care compositions. The pH-adjusting agent may be any compound or mixture of compounds that will achieve the desired pH. Suitable pH-adjusting agents include organic and inorganic acids and bases, such as benzoic acid, citric acid, potassium hydroxide, and sodium hydroxide. Buffering agents include acetate salts, borate salts, carbonate salts, bicarbonate salts (e.g., an alkali metal bicarbonate, such as sodium bicarbonate (also known as baking soda)), gluconates, tartrates, sulfates, citrates (such as sodium citrate), benzoate salts, nitrate salts (such as sodium and potassium nitrate), phosphate salts (such as potassium and sodium phosphate), and combinations of the foregoing as needed to achieve and maintain the desired pH.

Suitable antioxidants include superoxide dismutase, catalase, glutathione peroxidase, ebselen, glutathione, cysteine, N-acetyl cysteine, penicillamine, allopurinol, oxypurinol, ascorbic acid, α-tocopherol, Trolox (water-soluble α-tocopherol), vitamin A, β-carotene, fatty-acid binding protein, fenozan, probucol, cyanidanol-3, dimercaptopropanol, indapamide, emoxipine, dimethyl sulfoxide, and others.

Methods of contacting tissues of the mouth with oral care compositions are well known in the art. Suitable methods include rinsing the tissue with a solution (e.g., a mouthwash, rinse, spray, liquid dentifrice, or other solution), brushing the teeth with a dentifrice (e.g., a toothpaste, tooth gel, or powder), applying a non-abrasive solution, gel, paste, cream or ointment directly to the tissue (with or without the use of an applicator), chewing gum, chewing or sucking a lozenge, mint or tablet, and many other means of topical application. Suitable applicators for applying oral care compositions, such as solutions, gels, pastes, creams and ointments, to a tissue include a swab, a stick, a plastic paddle, a dropper, a syringe, a strip, a finger, or a dental tray or appliance which allows for immersion of the teeth and, optionally, the periodontal tissue in, e.g., a gel or solution. thereof. In some embodiments, oral care compositions further comprise, for instance, sutures can be used to close a surgical wound or a wound resulting from a tooth extraction, dental floss can be used to floss the teeth, etc. The treatment of the tissue can be prophylactic treatment. For instance, the tissue may be treated as part of a prophylactic oral care regimen.

Tissues may also be treated prophylactically in connection with a variety of dental procedures, including surgeries and tooth extractions. For instance, the tissue(s) on which surgery is being performed, those tissues near the area where the surgery is being performed or, for ease of treatment, all or substantially of the tissues of the mouth, can be treated prior to surgery, during surgery, after the surgery, or combinations thereof. Similarly for a tooth extraction, the tissue(s) surrounding the tooth which is to be extracted, adjacent tissues or, for ease of treatment, all or substantially of the tissues of the mouth, can be treated prior to tooth extraction, during the tooth extraction, after the tooth extraction, or combinations thereof. For instance, the mouth could be rinsed prior to surgery or tooth extraction with a solution comprising one or more cannabinoid as discussed herein, the wound(s) caused by the surgery or tooth extraction could be closed with sutures having one or more cannabinoid as discussed herein incorporated into them, and/or the mouth could be rinsed immediately after the surgery or tooth extraction, and/or at intervals thereafter, with a solution comprising one or more cannabinoid as discussed herein.

A composition comprising one or more cannabinoid as discussed herein can be used to treat a disease or condition of oral tissue. Diseases and conditions treatable according to the invention include inflammation and inflammatory disease and conditions, such as gingivitis and periodontitis. In some embodiments, conditions treated by oral care compositions comprising one or more cannabinoid as discussed herein may include periimplantitis, periodontitis, oral mucositis (especially oral mucositis caused by chemotherapy in cancer treatment), and dental pain. In some embodiments, a toothpaste, toothpowder or mouthwash comprising one or more cannabinoid as discussed herein, may be used in teeth brushing and/or rinsing at a therapeutically effective amount to treat one or more oral inflammatory condition. In some embodiments, the oral inflammatory condition is selected from periimplantitis, periodontitis, oral mucositis, and dental pain.

It is understood by those skilled in the art that the dosage amount of a one or more cannabinoid as discussed herein needed to treat a tissue of a subject's mouth will vary with the particular the type of oral care composition employed, whether the treatment is prophylactic or for the treatment of a disease or condition, the identity of the disease or condition to be treated, the severity of the disease or condition, the duration of the treatment, the identify of any other drugs being administered, the age, size and species of the animal, and like factors known in the medical and veterinary arts.

### EXAMPLES

The invention is further described in the following Examples. The Examples are merely illustrative and do not in any way limit the scope of the invention as described and claimed. This invention can be further illustrated by the following Examples of preferred embodiments thereof, although it will be understood that these Examples are included merely for purposes of illustration and are not intended to limit the scope of the invention unless otherwise specifically indicated.

Abbreviations used herein include the following:
DMEM Dulbecco's modified Eagle's medium
EGF Epidermal growth factor
ELISA Enzymelinked immunosorbent assay
FCS Fetal calf serum
GAM Goat anti-mouse
IL Interleukin
MW Molecular weight
NHEK Normal human epidermal keratinocytes
OD Optical density
PBS Phosphate buffered saline
PE Pituitary extract
PGE2 Prostaglandin E2
PMA Phorbol myristate acetate
ROS Reactive oxygen species
RT Room temperature
SFM Serum free medium
TGF Transforming growth factor
UV Ultraviolet
λem Emission wavelength
λex Excitation wavelength

### Example 1: Effects of Six Compounds on Normal Human Epidermal Keratinocytes

This example demonstrates the effects of compounds and L-Ascorbic on several skin models.

The effects on normal human epidermal keratinocytes (NHEK) were measured by assessing IL-8 and PGE2 release by PMA-stimulated NHEK using specific ELISA kits.

### Materials and Methods

### Biological Models

Normal human epidermal keratinocytes (NHEK):
Cell type: NHEK
Culture conditions: 37° C., 5% CO2
Culture medium: Keratinocyte SFM supplemented with Epidermal Growth Factor (EGF) 0.25 ng/ml
   Pituitary extract (PE) 25 μg/ml
   Gentamycin 25 μg/ml
Assay medium 1: Epilife® medium supplemented with PE 0.2%
   Bovine transferrin 5 μg/ml
   EGF 0.2 ng/ml
   Insulin 5 μg/ml
   Gentamycin/Amphotericin
Assay medium 2: Keratinocyte SFM supplemented with Gentamycin 25 μg/ml

### Test Compounds:

| | | | | | |
|---|---|---|---|---|---|
| CBG | Powder | 10 mM in | Systemic | NHEK, | 0.3, 1 and 3 μM |
| Purity: 98% | Storage: RT | ethanol | | NHDF | |
| FD200509-1 | | | | | |
| CBD | Powder | 10 mM in | Systemic | NHEK | 0.1, 0.3 and 1 |
| Purity: 99% | Storage: RT | ethanol | | | μM |
| FD200509-2 | | | | NDHF | 0.3, 1 and 3 μM |
| CBGVA | Powder | 10 mM in | Systemic | NHEK | 0.1, 0.3 and 1 |
| Purity: 97% | −20° C. | ethanol | | | μM |
| FD200509-3 | Storage: | | | NHDF | 1, 3 and 10 μM |
| CBDA | Powder | 10 mM in | Systemic | NHEK | 0.03, 0.1 and 0.3 |
| Purity: 97% | −20° C. | ethanol | | | μM |
| FD200509-4 | Storage: | | | NHDF | 1, 3 and 10 μM |
| CBGA | Powder | 10 mM in | Systemic | NHEK | 0.03, 0.1 and 0.3 |
| Purity: 97% | −20° C. | ethanol | | | μM |
| FD200509-5 | Storage: | | | NHDF | 1, 3 and 10 μM |
| L-Ascorbic | Powder | 1 mg/ml in | Systemic | NHEK, | 50, 100 and 200 |
| acid | Storage: RT | culture | | NHDF | μg/ml |
| Ref. Sigma, ref. | protected | medium, | | | |
| A4544 Batch n^{o} | from light | assay | | | |
| SLBX2310 | and humidity | medium or | | | |
| MW: 176.12 | | PBS | | | |
| FD200509-6 | | | | | |

### IL-8 and PGE2 Release by PMA-Stimulated NHEK

### Culture and Treatments

Keratinocytes were seeded in 96-well plates and cultured for 24 hours in culture medium. The medium was then replaced by assay medium 1 containing or not (control) the test compounds or the reference compound (staurosporine tested at 1 nM for IL-8 release or indomethacin tested at 1 μM for PGE2 release) and the cells were pre-incubated for 24 hours. After pre-incubation, the medium was removed and replacedby assay medium 1 containing or not (stimulated control) the compounds or the reference compound and containing the inducer (PMA tested at 0.5 μg/ml). The cells were then incubated for 24 hours. In parallel, a non-stimulated control condition was performed.

All experimental conditions were performed in n=3. At the end of incubation, the culture supernatants were collected for IL-8 and PGE2 quantifications.

### Enzyme-Linked Immunosorbent Assay (ELISA)

IL-8 and PGE2 released in the culture supernatants were measured using specific ELISA kits according to the supplier's instructions.

| | | Low limit of | High limit of |
|---|---|---|---|
| Kit | Supplier | detection | detection |
| Human IL- | R&D SystemsRef. DY208 | 31.25 pg/ml | 2000 pg/ml |
| Duo Set | Dilution factor: 1/10 | 312.5 pg/ml | 20,000 pg/ml |
| 8/CXCL8 | Enzo Ref. ADI-900-001 | 39.1 pg/ml | 2,500 pg/ml |
| PGE2 | | | |

### Data Management

Raw data were analyzed using Microsoft Excel® software and GraphPad PRISM® software.

The inter-group comparisons were performed by an unpaired Student's t-test. The statistical analysis can be interpreted if n≥5, however for n<5 the statistical values are for information only.

### Results

### Effect on IL-8 Release by PMA-Stimulated Keratinocytes

The effects of compounds and of L-ascorbic acid on IL-8 released are summarized in Table 1.

TABLE 1
Effect of compounds and L-Ascorbic acid on IL-8 release
by PMA-stimulated keratinocytes.

| | | Treatment | Normalized data |
|---|---|---|---|
| | Test compound | Concentration | % Relative inhibition |
| Stimulated | Non-stimulated | | 100 |
| conditions: | control* | | |
| PMA-0.5 | Control | — | 0 |
| μg/ml | Staurosporinel | 1 μM | 68 |
| | CBG | 0.3 μM | 7 |
| | | 1 μM | 20 |
| | | 3 μM | 60 |
| | CBD | 0.1 μM | 12 |
| | | 0.3 μM | −44 |
| | | 1 μM | 18 |
| | CBGVA | 0.1 μM | 2 |
| | | 0.3 μM | 11 |
| | | 1 μM | 9 |
| | CBDA | 0.03 μM | 11 |
| | | 0.1 μM | 17 |
| | | 0.3 μM | 0 |
| | CBGA | 0.03 μM | 0 |
| | | 0.1 μM | −7 |
| | | 0.3 μM | 9 |
| | L Ascorbic | 50 μg/ml | −10 |
| | acid | 100 μg/ml | 11 |
| | | 200 μg/ml | −15 |

In the non-stimulated control condition, a low basal release of IL-8 was detected in normal human epidermal keratinocytes (NHEK) (80 pg/ml). The treatment of NHEK with PMA at 0.5 μg/ml for 24 hours highly induced IL-8 release (2406 pg/ml) and this effect was significantly inhibited by the reference compound staurosporine tested at 1 nM (68% of relative inhibition). These results were expected and validated the assay.

Under the experimental conditions of this study, CBG, when tested at 3 strongly inhibited the release of IL-8 by PMA-stimulated keratinocytes.

None of the other tested compounds were able to revert PMA stimulation. On the contrary, CBD overstimulated IL-8 release by the keratinocytes when tested at 0.3 μM but this effect was not validated when the compound was tested at other concentrations.

### Effect on PGE2 Release by PMA-Stimulated Keratinocytes

The effects of compounds and of L-ascorbic acid on PGE2 release are summarized in Table 2.

TABLE 2
Effect of compounds and L-Ascorbic acid on PGE2
release by PMA-stimulated keratinocytes

| | | Treatment | Normalized data |
|---|---|---|---|
| | Test compound | Concentration | % Relative inhibition |
| Stimulated | Non-stimulated | | 100 |
| conditions: | control | | |
| PMA-0.5 | Control | — | 0 |
| (μg/ml) | Indomethacin | 1 μM | >169 |
| | CBG | 0.3 μM | 86 |
| | | 1 μM | >169 |
| | | 3 μM | >159 |
| | CBD | 0.1 μM | 0 |
| | | 0.3 μM | −59 |
| | | 1 μM | −242 |
| | CBGVA | 0.1 μM | 19 |
| | | 0.3 μM | 65 |
| | | 1 μM | >162 |
| | CBDA | 0.03 μM | −24 |
| | | 0.1 μM | −50 |
| | | 0.3 μM | −20 |
| | CBGA | 0.03 μM | −17 |
| | | 0.1 μM | −68 |
| | | 0.3 μM | −233 |
| | L Ascorbic | 50 μg/ml | >148 |
| | acid | 100 μg/ml | >160 |
| | | 200 μg/ml | 127 |

In the non-stimulated control condition, a low basal level of PGE2 was secreted by NHEK (62 pg/ml). The treatment of NHEK with PMA at 0.5 μg/ml for 24 hours resulted in a significant stimulation of PGE2 release (96 pg/ml) and this effect was completely inhibited by the reference compound indomethacin, tested at 1 μM (>169% of relative inhibition). These results were expected and validated the assay.

CBG, CBGVA, and L-Ascorbic acid strongly and significantly inhibited PGE2 release by PMA-stimulated keratinocytes (about 160%, 160% and 145% of relative inhibition, respectively). Regarding CBG, the inhibitory effect was significant at all tested concentrations and equivalent when tested at 1 and 3 μM. CBGVA had a significant effect only when tested at the highest concentration. Finally, L-Ascorbic acid induced overall the same effect at all tested concentrations.

On the other hand, CBD and CBGA overstimulated PGE2 release by PMA-stimulated keratinocytes reaching respectively 185% and 182% of the control when tested at the highest concentration. Finally, CBDA did not modulate PGE2 release by PMA-stimulated keratinocytes.

### CONCLUSIONS

In the conditions of this study, L-Ascorbic acid significantly limited PGE2 release from PMA-stimulated keratinocytes

CBG significantly inhibited the release of IL-8 and PGE2 from PMA-stimulated keratinocytes.

CBGVA inhibited PGE2 release from PMA-stimulated keratinocytes.

As used herein, all percentages (%) are percent by weight of the total composition, also expressed as weight/weight %, % (w/w), w/w, w/w % or simply %, unless otherwise indicated.

The dimensions and values disclosed herein are not to be understood as being strictly limited to the exact numerical values recited. Instead, unless otherwise specified, each such dimension is intended to mean both the recited value and a functionally equivalent range surrounding that value.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in the document shall govern.

The foregoing description of embodiments and examples has been presented for purposes of description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein, but can be employed in any number of applications and equivalent articles by those of ordinary skill in the art. Rather it is hereby intended the scope be defined by the claims appended hereto.

What is claimed is:

1. An oral care product to reduce, treat, prevent, or ameliorate oral inflammation, comprising one or more cannabinoid compounds comprising cannabigerol (CBG) and cannabigerovarinic acid (CBGVA).

2. The oral care product of claim 1 is a toothpaste, a mouthwash, a chewable, a wipe, a rinse, a powder, or a floss.

3. The oral care product of claim 1 comprises about 0.01 μg/mL to about 1 mg/mL of the one or more cannabinoid compounds.

4. The oral care product of claim 1 further comprises one or more additional oral care active ingredients.

5. The oral care product of claim 4, wherein the one or more additional oral care active indigents comprise stannous fluoride, essential oil, and fluoride ions.

6. The oral care product of claim 4, wherein the one or more additional oral care active indigents comprise fluoride ions.

7. The oral care product of claim 1 prevents or reduces the release of interleukin 8 (IL-8).

8. The oral care product of claim 1 prevents or reduces the release of prostaglandin E2 (PGE2).

9. The oral care product of claim 1 prevents or reduces the release of both IL-8 and PGE2.

10. The oral care product of claim 1 reduces one or more conditions associated with gingivitis, periodontal disease, periimplantitis, oral mucositis, chemotherapy-induced oral mucositis, and dental pain.

11. A method of making an oral care product comprising adding one or more cannabinoid compounds to an oral care formulation, and
wherein the one or more cannabinoid compounds comprise cannabigerol (CBG) and cannabigerovarinic acid (CBGVA).

12. The method of claim 11, wherein the one or more cannabinoid compounds reduce oral inflammation.

13. The method of claim 12, wherein the oral inflammation is associated with or results in gingivitis, periodontal disease, periimplantitis, oral mucositis, chemotherapy-induced oral mucositis, and/or dental pain.

14. The method of claim 11, wherein the one or more cannabinoid compounds decrease the release of interleukin 8 (IL-8), prostaglandin E2 (PGE2), or both in an oral cavity.

15. A method of reducing interleukin 8 (IL-8), prostaglandin E2 (PGE2), or both, in an oral cavity, comprising contacting the oral cavity with an effective amount of one or more cannabinoid compounds; and
wherein the one or more cannabinoid compounds comprise cannabigerol (CBG) and cannabigerovarinic acid (CBGVA).
